(19) United States
(12) Patent Application Publication
(10) Pub. No.: US 2005/0256436 A1
(43) Pub. Date: Nov. 17, 2005

(54) Medical immobilizing device and method

(76) Inventors: Jenet Irene Powell, Canton, OH (US); Jason Michael Skaarup, Centreville, VA (US)

Correspondence Address: Janet Irene Powell, 1639 Elberta St. SW, Canton, OH, 44709, US

(21) Appl. No.: 10/846,748

(22) Filed: May 14, 2004

(51) Int. Cl. A61F 5/00

(52) U.S. Cl. 602/4

(57) ABSTRACT

A medical immobilizing device and method for immobilizing at least one arm of a person are provided. The device includes an axial strap configured to be secured around a torso of the person and an arm carrying member operatively associated with the axial strap. The arm carrying member is constructed and arranged to support a portion of at least one arm of the person when the axial strap is secured to the torso of the person. The method includes providing an axial strap that is configured to be secured around a torso of the person and providing an arm carrying member that is operatively associated with the axial strap to support a portion of at least one arm of the person.

### BACKGROUND OF THE INVENTION

1. Field of the Invention

The invention relates to a medical immobilizing device and method that can stabilize or immobilize at least one arm of a person.

2. Description of Related Art

Conventionally, orthopedic medicine extensively uses arm slings or arm immobilizers whenever a person must stabilize or immobilize his or her arm, generally following an injury or illness. Typical injuries or illnesses that require arm slings or arm immobilizers can include shoulder fractures, hemiplegia or other stroke related injuries, bursitis, rotator cuff injuries, Rheumatoid arthritis, clavicle fractures and dislocations, acromio-mastoid and brachial-plexus injuries, arcomio-clavicular sprains, dislocation of the elbow and fractures of the humerus, radius and ulna (arm). Other injuries or sickness could also require arm slings or arm immobilizers.

Such arm slings or arm immobilizers generally have an arm carrying sleeve into which the person's arm can be placed so that one of the person's hands extends partially or fully outside the arm carrying sleeve. A shoulder strap is typically secured to the arm carrying sleeve of the arm sling or arm immobilizer and positioned around and over the person's shoulder or neck so that the weight of the arm is supported by the arm carrying sleeve and the shoulder strap while the arm is kept in a protected or supported manner. However, the strap of conventional arm slings or arm immobilizers can cause undue pressure on the person's upper body, particularly their shoulders or neck, for example, when the shoulder strap is positioned over the person's shoulder or neck and carries the weight of the person's arm.

### SUMMARY OF THE INVENTION

One aspect of the invention is to provide a medical immobilizer device that can protect, support, retain or position an arm of a person wearing the medical immobilizer device.

Another aspect of the invention is to provide a medical immobilizer device for a person comprising an axial strap having first and second ends configured to be releasably secured around a torso of the person. The medical immobilizer device also includes an arm carrying member operatively associated with the axial strap. The arm carrying member is constructed and arranged to support a portion of an arm of the person when the axial strap is secured to the person's torso.

Yet another aspect of the invention is to provide a medical immobilizer device configured for use with an adjustable belt having first and second ends. The medical immobilizer device comprises a support structure removably mounted to the adjustable belt and an arm carrying member removably mounted to the support structure and constructed and arranged to support a portion of an arm of the person.

Another aspect of the invention is to provide a method of immobilizing an arm of a person. The method comprises positioning an axial strap around a torso of the person and supporting a portion of the arm with an arm carrying member operatively associated with the axial strap.

### BRIEF DESCRIPTION OF THE DRAWINGS

The invention will be described in conjunction with the following drawings in which like reference numerals designate like elements and wherein:

FIG. 1 is a perspective view of a medical immobilizer device in an operative position on a person in accordance with the principles of the present invention;

FIG. 2 is a perspective view of the medical immobilizer device shown in FIG. 1;

FIG. 3 is a top view of the medical immobilizer device shown in FIG. 1, but with the device in an inoperative position;

FIG. 4 is a cross-sectional view taken through line 4-4 of FIG. 3 showing one arm carrying member in greater detail;

FIG. 5 is a perspective view of another embodiment of a medical immobilizer device in accordance with the principles of the present invention;

FIG. 6 is a front view of yet another embodiment of a medical immobilizer device in accordance with the principles of the present invention;

FIG. 7 is a front view of the medical immobilizer device shown in FIG. 5, but with an arm carrying member thereof pivoted relative to the device;

FIG. 8 is a perspective view of another embodiment of a medical immobilizer device in accordance with the principles of the present invention;

FIG. 9 is a perspective view of the medical immobilizer device shown in FIG. 8;

FIG. 10 is a perspective view of the medical immobilizer device shown in FIG. 8, but in an operative position on a person;

FIG. 11 is a top view of a support structure and arm carrying members for use with a transfer belt in accordance with the principles of the present invention;

FIG. 12 is a top view of the support structure shown in FIG. 11 without the transfer belt and arm carrying members;

FIG. 13 is a top view of two support structures shown in FIG. 12 and arm carrying members in accordance with the principles of the present invention;

FIG. 14 is a top view of another embodiment of the support structure shown in FIG. 11 in accordance with the principles of the present invention;

FIG. 15 is a top view of another embodiment of the support structure shown in FIG. 13 in accordance with the principles of the present invention; and

FIG. 16 is a flow chart illustrating a method of immobilizing an arm of a person in accordance with the present invention.

### DETAILED DESCRIPTION OF EMBODIMENTS

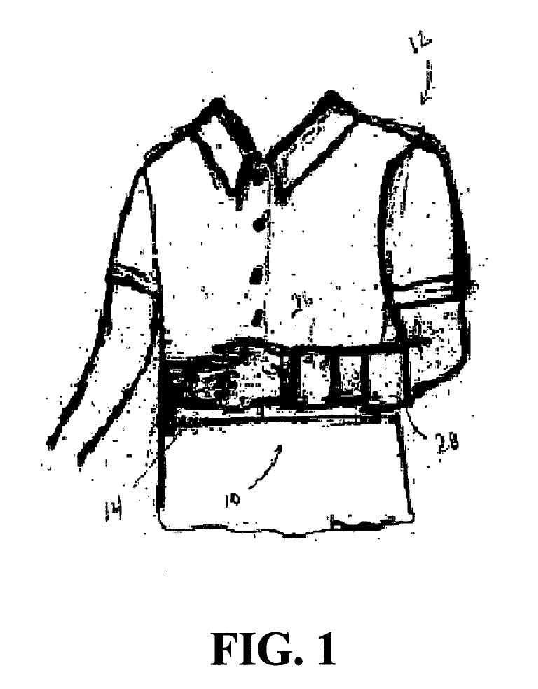

FIG. 1 shows a medical immobilizer device, generally indicated at 10, being worn by a person 12. The medical immobilizer device 10 can protect, support, retain or position an arm of the person 12 wearing the medical immobilizer device 10, generally after the person 12 suffers injury or sickness. The arm of the person 12 can be positioned alongside the person's umbilicus in FIG. 1, but can be positioned alongside other areas of the person's torso as well, for example, the person's chest or rib area.

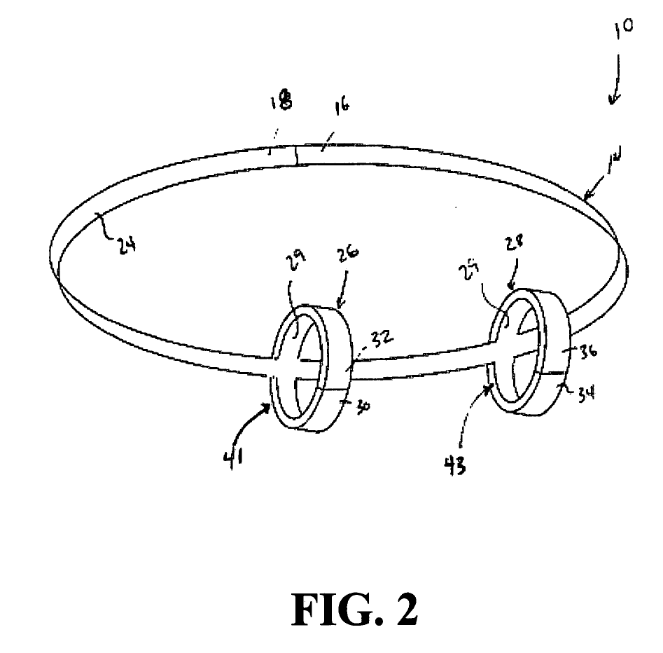

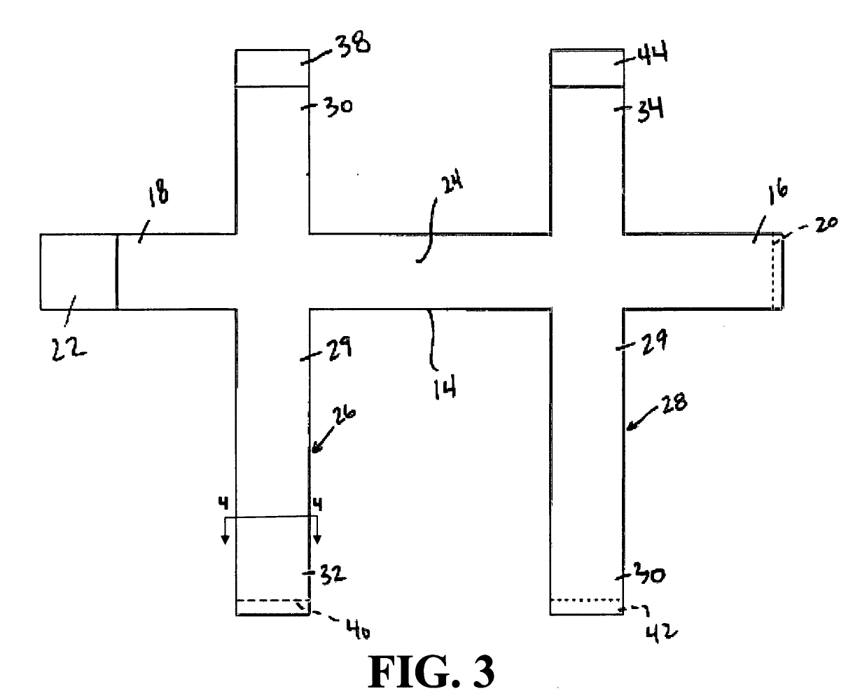

FIGS. 2 and 3 show the medical immobilizer device 10 in greater detail. The medical immobilizer device 10 includes an axial strap 14 that has a first end 16 and a second end 18. The first and second ends 16, 18 have respective cooperating fastening structure 20, 22 to be releasably secured around a torso of the person 12. The cooperating fastening structure 20, 22 may include hook and loop fasteners, cooperating interlocking members, such as a buckle or snap, or any other fastening structure capable of securing the first and second ends 16, 18 around a torso of the person 12. As shown in FIGS. 2 and 3, the cooperating fastening structure 20 constitutes a loop fastener and the cooperating structure 22 constitutes a hook fastener configured to engage the loop fastener 20. The loop fastener 20 can have a larger surface area than the hook fastener 22, which allows for size adjustment of the axial strap 14 depending on the size of the person 12. For example, the hook fastener 22 can engage an outermost portion of the loop fastener 20 when secured to a person with a large torso and the hook fastener 22 can engage an innermost portion of the loop fastener 20 when secured to a person with a small torso. Thus, the axial strap 14 can be adjusted for different sized persons.

The axial strap 14 can be formed of any flexible, substantially rigid material capable of supporting a person's arm or arms in the medical immobilizer device 10. Such flexible, substantially rigid materials can include cloth or plastic, for example. The axial strap 14 can also be a transfer belt, such as a gait belt, that is commonly secured around the torso of medical patients having mobility difficulties, for example. A padded interior surface 24 can extend from the first end 16 to the second end 18 of the axial strap 14. The padded interior surface 24 can be sponge, foam, rubber, an air filled pocket, a compressible material or other padding material, for example, to provide desired comfort for the person 12 when engaged with the torso of the person 12.

Arm carrying members 26, 28 can integrally extend from the axial strap 14 in a direction transverse thereto, for example, as shown in FIG. 3. The arm carrying members 26, 28 can be constructed and arranged to support a portion of an arm of the person 12 when the axial strap 14 is secured to the person's torso and when moved into an operative position thereof. Each arm carrying member 26, 28 can be of identical construction and can be formed of flexible, substantially rigid material 33, such as cloth or plastic, for example, and a padded material 31. The padded material 31 can be sponge, foam, rubber, an air filled pocket, a compressible material or other padding material, for example, to provide desired comfort for the person 12 when engaged with the torso of the person 12.

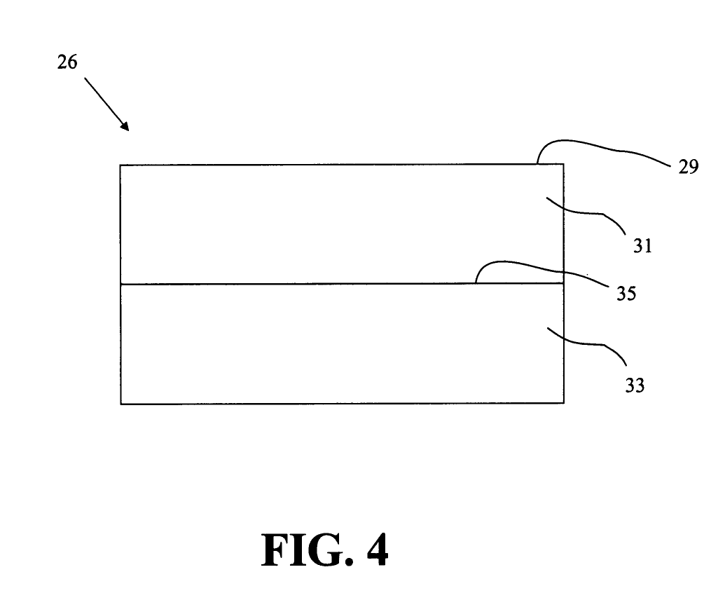

FIG. 4 shows a cross-sectional view of arm carrying member 26. Although arm carrying member 26 is shown in FIG. 4, the cross-sectional view therein can also be representative of arm carrying member 28 since both arm carrying members 26, 28 have identical structure. The axial strap 14 can have identical structure as the arm carrying members 26, 28 as well. The padded material 31 includes a padded interior surface 29 configured to engage an arm of the person 12. The padded material 31 can be secured to the substantially rigid material 33 by stitching 35, for example. Alternatively, the padded material 31 can be secured to the substantially rigid material 33 with bonding, fasteners, adhesives or other securing means, for example, which could replace the stitching 35. When the arm carrying member 26, 28 is positioned around an arm of the person 12, in an operative position, the padded interior surface 29 engages the arm of the person 12.

Each arm carrying member 26, 28 has end sections 30, 32 and 34, 36, respectively, as shown in FIGS. 2, 3 and 4. The padded interior surface 29 can extend between respective end sections 30, 32 and 34, 36 of the arm carrying members 26, 28. The end sections 30, 32 have respective cooperating fastening structure 38, 40 to releasably secure the arm carrying member 26 around a portion of the person's arm. The cooperating fastening structure 38, 40 may include hook and loop fasteners, cooperating interlocking members, such as a buckle or snap, or any other fastening structure capable of securing the end sections 30, 32 around the person's arm. As shown in FIG. 3, the cooperating fastening structure 38 constitutes a loop fastener and the cooperating structure 40 constitutes a hook fastener configured to engage the loop fastener 38. When the end sections 30, 32 or 34, 36 of respective arm carrying members 26, 28 engage one another, an arm supporting opening 41 (FIG. 2) is provided for supporting or receiving a portion of the person's arm therethrough. The operative position of the arm carrying members 26, 28 is defined when the respective end sections 30, 32 or 34, 36 thereof engage one another to support a portion of the person's arm. FIG. 1 shows the arm carrying member 26 secured around a wrist portion of the person 12 (not shown), for example, such that the padded interior surface 29 would contact the wrist portion.

The end sections 34, 36 have respective cooperating fastening structure 42, 44 to releasably secure the arm carrying member 28 around a portion of the person's arm. The cooperating fastening structure 42, 44 may include hook and loop fasteners, cooperating interlocking members, such as a buckle or snap, or any other fastening structure capable of securing the end sections 34, 36 around the person's arm. As shown in FIG. 3, the cooperating fastening structure 42 constitutes a loop fastener and the cooperating structure 44 constitutes a hook fastener configured to engage the loop fastener 42. When the end sections 34, 36 of arm carrying member 28 engage one another, an arm supporting opening 43 (FIG. 2) is provided for receiving a portion of the person's arm therethrough. FIG. 1 shows the arm carrying member 28 secured around a forearm portion of the person 12, for example.

Rigid, flexible elements formed of thin sheet metal or some other rigid, flexible material, for example, can be positioned beneath the padded interior surface 29 or the padded material 31 of each arm carrying member 26, 28. The rigid, flexible elements can be used to substantially rigidify the arm supporting opening 41, 43 of each arm carrying member 26, 28 by conforming to the shape of the person's arm when the person's arm extends through the arm supporting opening of each arm carrying member 26, 28. In this manner, the arm carrying members 26, 28 can be sufficiently rigid to space the person's arm from the person's torso to facilitate comfort in certain applications, for example.

FIG. 1 shows a left arm of the person 12 secured near the person's torso with the medical immobilizer device 10, but the arm carrying members 26, 28 are configured to receive either the left arm or the right arm of the person 12. To effect application to the right arm of the person 12, the axial strap 14 can be adjusted, e.g., rotated about the person's torso, so that the arm carrying member 26 engages the forearm portion of the person's right arm and the arm carrying member 28 engages the wrist portion of the person's right arm, for example.

Although two arm carrying members 26, 28 are shown in FIGS. 1-3, one arm carrying member is sufficient to support a portion of the person's left or right arm. When only one arm carrying member is provided in the medical immobilizer device 10, the rigid, flexible element 31 may be included to provide additional support to the person's left or right arm. Additionally, the width of the arm carrying member may be increased to distribute the support forces over the arm more evenly. In instances where only one arm carrying member is provided in the medical immobilizer device 10, the arm carrying member may engage either the wrist or forearm portion of the person's arm.

In some medical applications, such as for stroke victims, both arms of the person 12 can be supported and restrained by the arm carrying members 26, 28. To effect application to both arms of the person, each arm carrying member 26, 28 is positioned to engage both arms of the person 12 simultaneously. For example, the arm carrying member 26 can engage the wrist of the left arm of the person and the forearm of the right arm of the person 12 while the arm carrying member 28 can engage the wrist of the right arm of the person 12 and the forearm of the left arm of the person 12.

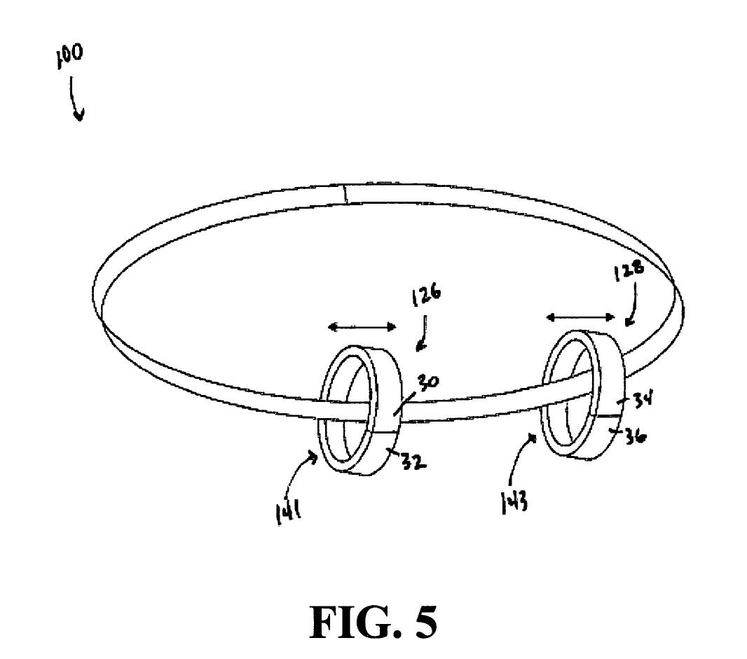

FIG. 5 shows another medical immobilizer device 100, which is an alternative embodiment of the device 10. In the following description of device 100, only the points of difference of the device 100 from the device 10 illustrated in FIGS. 1-4 will be described. That is, in this embodiment, the constituent parts the same as those in the embodiment illustrated in FIGS. 1-4 are referenced correspondingly in the drawings and the description about them will be omitted. The device 100 operates in a substantially similar manner as the device 10, but realizes different construction.

The arm carrying members 126, 128 are of the same construction as the arm carrying members 26, 28 and further description of the arm carrying members 126, 128 will be omitted. Arm carrying members 126, 128 of device 100 are movably attached to the axial strap 14 instead of being integral with the axial strap 14 (as indicated by double ended arrows). For example, the arm carrying members 126, 128 of device 100 are movably attached to the axial strap 14 through the respective arm supporting openings 141, 143 thereof. The arm supporting openings 141, 143 are identical in construction and operation as the arm supporting openings 41, 43 described above.

When the end sections 30, 32 and 34, 36 are not engaged with one another, the arm carrying members 126, 128 can be removed from the axial strap 14. Removal of the arm carrying members 126, 128 can facilitate adjustment of the arm carrying members 126, 128 by positioning the arm carrying members 126, 128 to different portions of different people's arms for each application, for example. When the end sections 30, 32 and 34, 36 are engaged with one another, the arm carrying members 126, 128 are movably attached to the axial strap 14 through the respective arm supporting openings thereof. The movable attachment allows the arm carrying members 126, 128 to be positioned anywhere along the axial strap 14, which allows for size adjustment for each application to arms of different people. For example, the arm carrying members 126, 128 can be moved relative to the axial strap 14 to engage wrist or forearm portions of people with different sized arms.

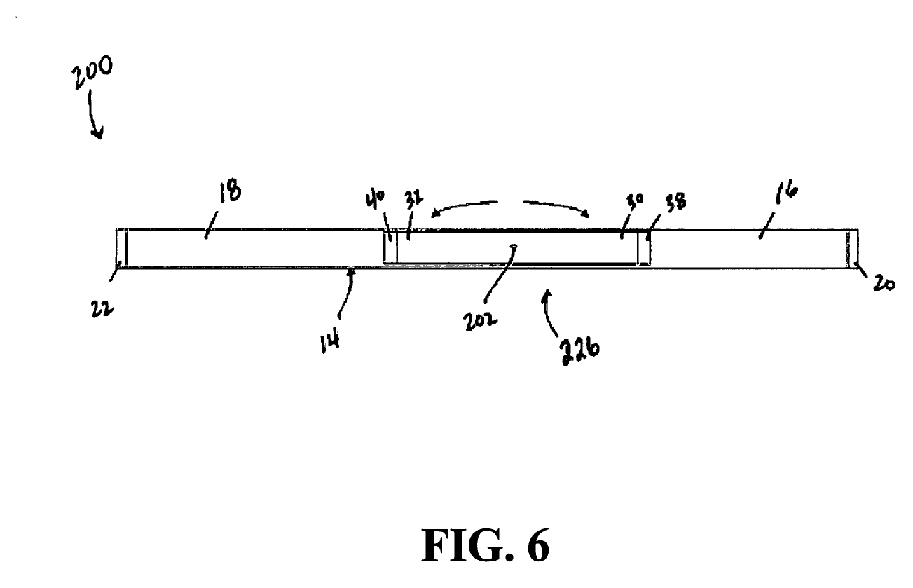

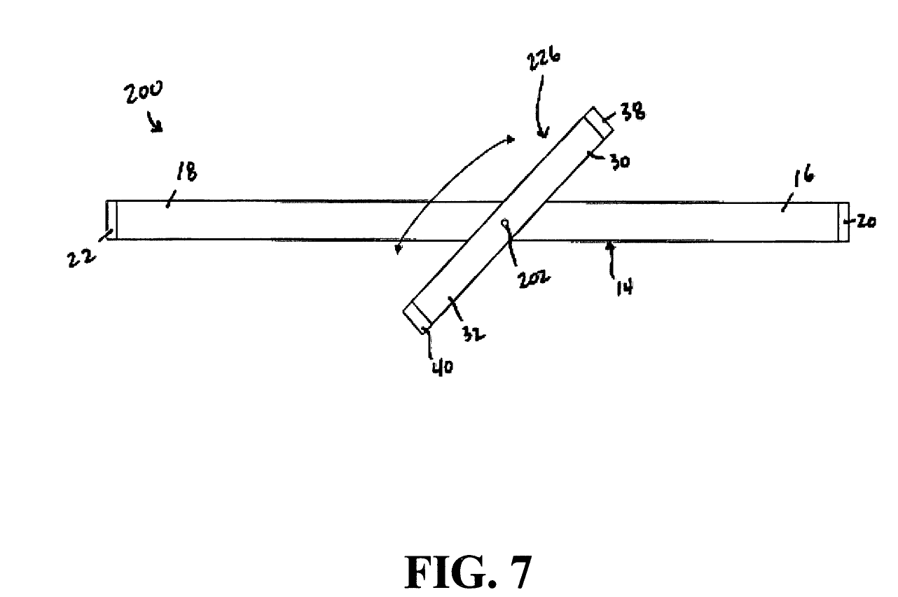

FIGS. 6 and 7 show another medical immobilizer device 200, which is an alterative embodiment of the device 10. The device 200 includes the axial strap 14 and one arm carrying member 226. The arm carrying member 226 is of the same construction as the arm carrying members 26, 28, 126, 128 and further description of the arm carrying member 226 will be omitted.

The arm carrying member 226 is pivotally attached to the axial strap 14 via a fastener 202. The fastener 202 can include a rivet or some other fastener that allows the arm carrying member 226 to pivot relative to the axial strap 14 (as indicated by arrows). FIG. 6 shows the arm carrying member 226 pivoted substantially parallel to the axial strap 14, while FIG. 7 shows the arm carrying member 226 pivoted at an angle relative to the axial strap 14. The pivotal movement of the arm carrying member 226, in either a clockwise or counter-clockwise direction and at any angle, provides additional flexibility for positioning the left or right arm of the person 12 in the device 200. The arm carrying member 226 can include a flexible, rigid element (not shown) as described above.

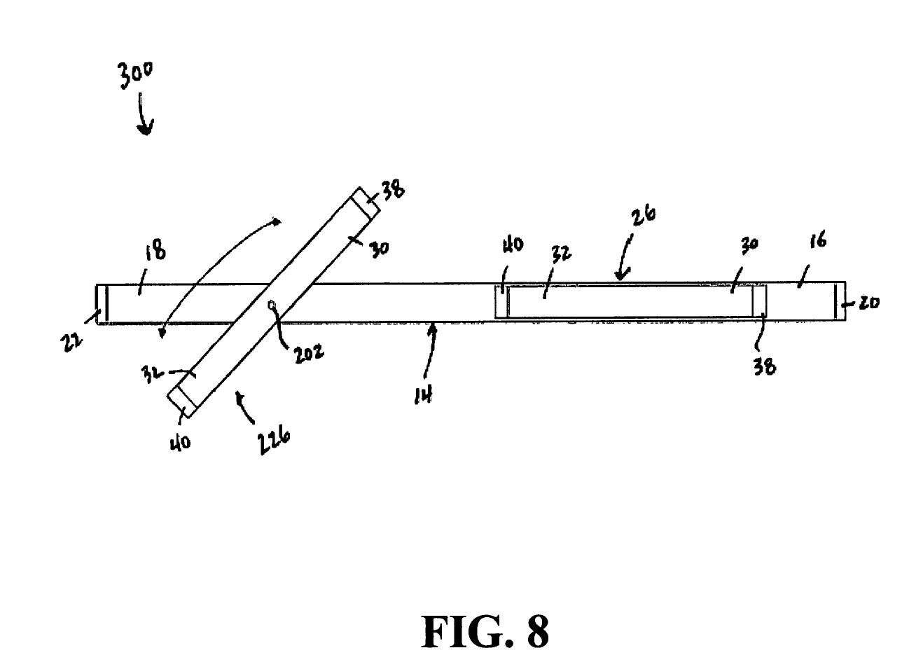

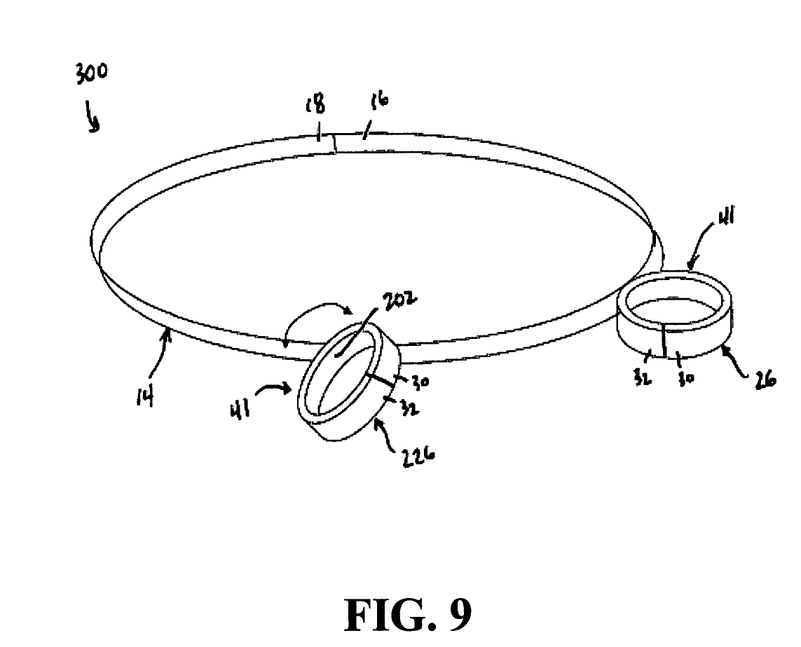

FIGS. 8, 9 and 10 show another medical immobilizer device 300, which is an alterative embodiment of the device 200. The device 300 includes the axial strap 14, the arm carrying member 226 and one arm carrying member 26. Any one of arm carrying members 28, 126 or 128 could be used with the arm carrying member 226 because the arm carrying members 28, 126 and 128 have identical construction as the arm carrying member 26.

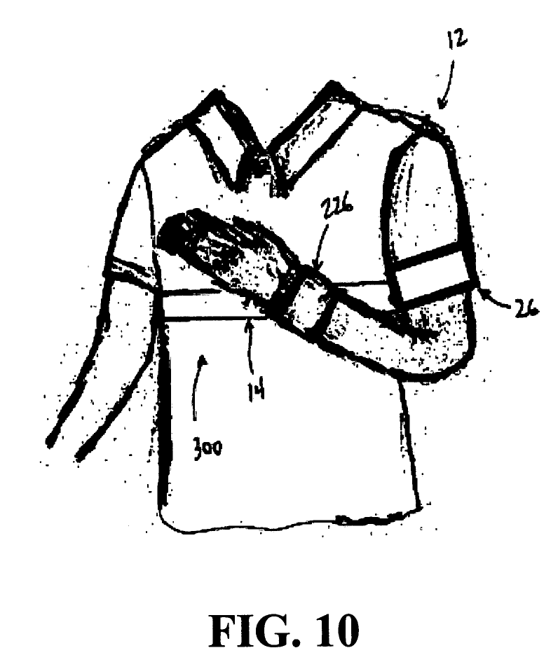

As shown in FIG. 10, the axial strap 14 is positioned around an upper torso of the person 12, e.g., near the person's chest or rib area. The arm carrying member 226 is pivoted relative to the axial strap 14 to engage a wrist portion of the person's left arm. The one arm carrying member 26 engages a bicep portion of the person's left arm to further secure the person's arm in the device 300. Although the device 300 is shown in FIG. 10 supporting the person's left arm, the device 300 is equally capable of supporting the person's right arm.

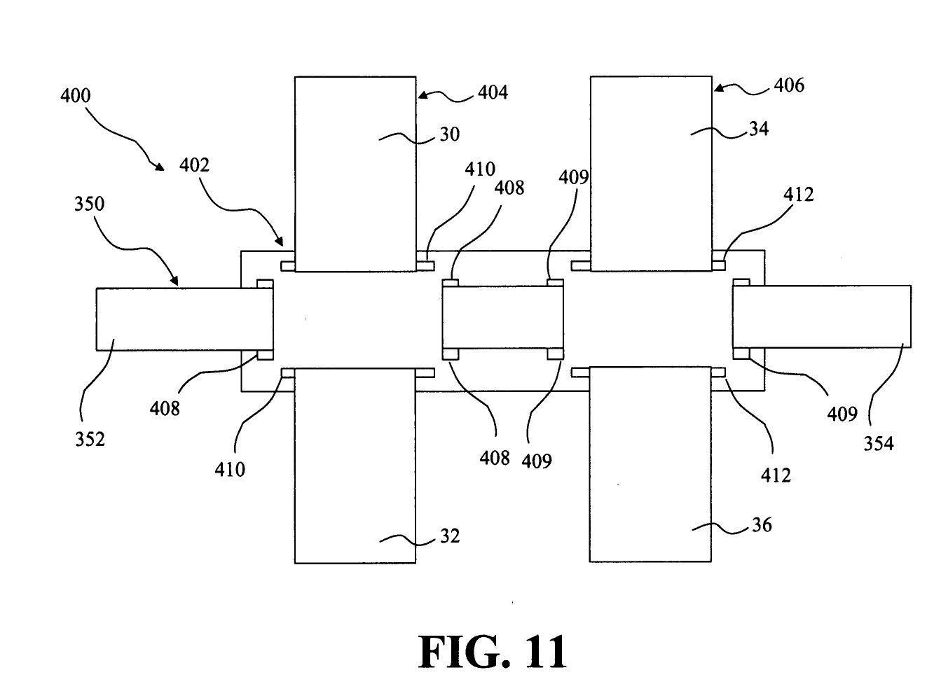

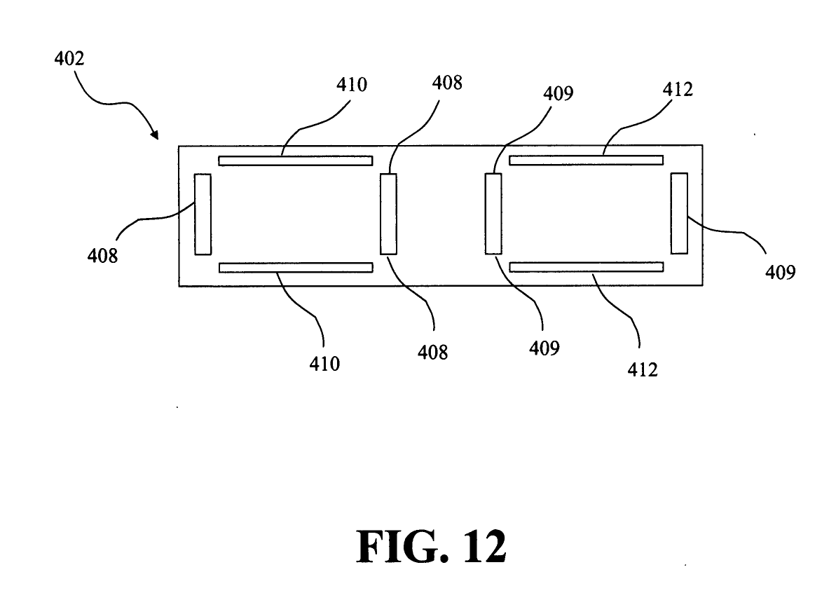

FIG. 11 shows a conventional transfer belt 350, also known as a gait belt, which is commonly secured around the waist of invalids and other medical patients having mobility difficulties. The transfer belt 350 allows a medical assistant or care taker to assist the movement of the patient by providing a belt which can be grasped by the assistant or care taker. The transfer belt 350 has first and second ends 352, 354 having an adjustable closure assembly (not shown), such as a buckle, attached thereto. The adjustable closure assembly can allow for adjustment of the transfer belt 350 for patients of different sizes. FIG. 11 also shows a medical immobilizer device 400 configured for use with the conventional transfer belt 350. The device 400 includes a support structure 402 (FIGS. 11 and 12) configured to be movably attached to the transfer belt 350 and two arm carrying members 404, 406 removably attached to the support structure for supporting the person's left or right arm.

FIG. 12 shows the support structure 402 in greater detail. The support structure 402 can be formed of sufficiently rigid construction, for example, from steel, plastic, rubber or another sufficiently rigid material. The support structure 402 includes a series of openings 408 and a series of openings 409 through which the transfer belt 350 can be positioned to secure the support structure 402 to the transfer belt 350. As shown in FIG. 11, the transfer belt 350 is positioned through the openings 408, 409 so that the support structure 402 is slidably engaged with the transfer belt 350 for axial movement along the length of the transfer belt 350. The support structure 402 is secured against the torso of the person 12.

As illustrated in FIGS. 11 and 12, the support structure 402 also includes a first pair of openings 410 and a second pair of openings 412 through which the arm carrying members 404, 406 can be attached, respectively. The arm carrying member 404 extends through the pair of openings 410 and the arm carrying member 406 extends through the pair of openings 412 so that each arm carrying member 404, 406 is engaged with the support structure 402. The arm carrying members 404, 406 are removable from the support structure 402 for replacement, if necessary. Also, arm carrying members of different lengths can be substituted for one or both of the arm carrying members 404, 406 depending on the intended application.

The distances between respective openings 408 and 409 and the distances between respective openings 410 and 412 and the respective sizes thereof can be adjusted as needed to accommodate for arms or arm carrying members of different sizes, for example. For example, the distance between the first series of openings 408 and the second series of openings 409 could be increased for a person having a longer arm to provide sufficient support for the person's arm. Similarly, the distance between the openings 410 and the distance between the openings 412 could be increased respectively for a person having an arm with large diameter, for example.

The arm carrying members 404, 406 can be of identical construction and operation as the arm carrying members 126, 128, for example, and can have respective ends 30, 32 and 34, 36.

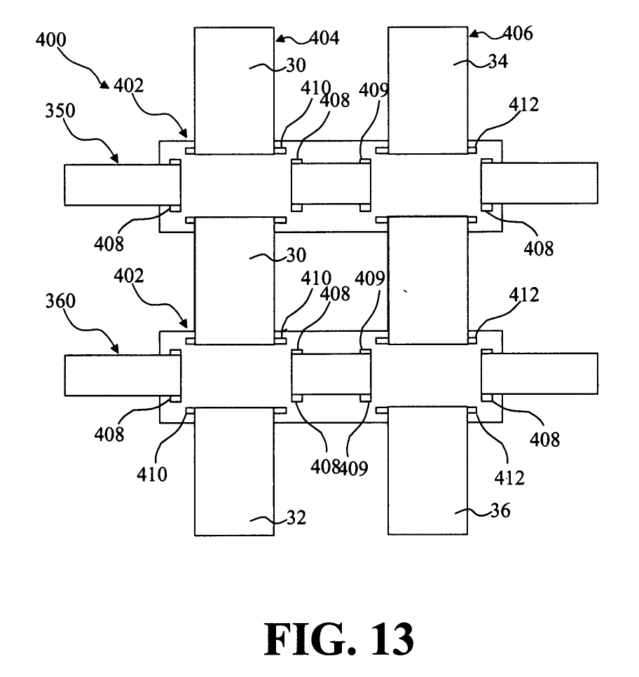

FIG. 13 shows two support structures 402 connected together in spaced relation by the arm carrying members 404, 406 to support both arms of the person 12. For example, Separate transfer belts 350, 360 could be positioned through openings 408, 409 of each respective support structure 402 and each arm carrying member 404 and 406 would be received through the respective openings 410, 412 of each respective support structure 402, for example.

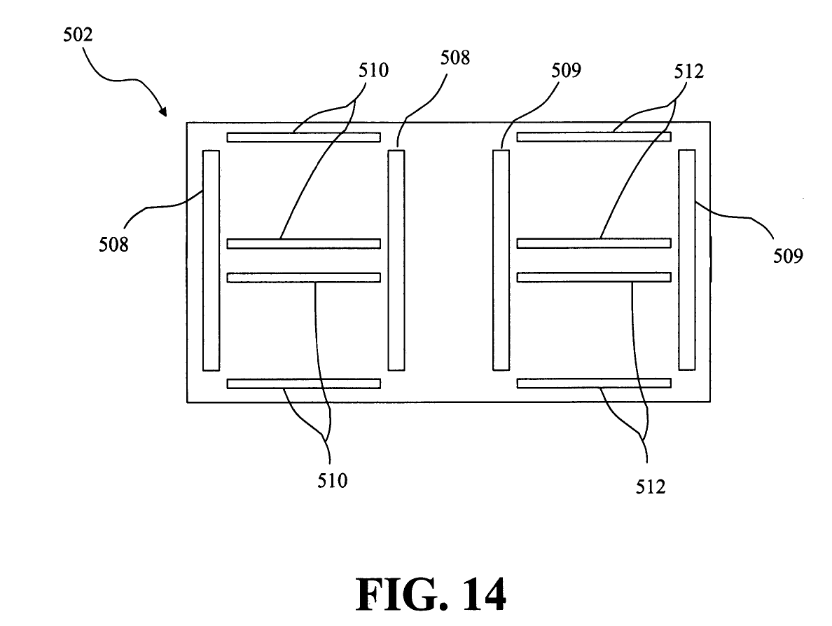

FIG. 14 shows another support structure 502, which is an alternative embodiment of the support structure 402. In the following description of support structure 502, only the points of difference of the support structure 502 from the support structure 402 illustrated in FIGS. 11 and 12 will be described. The support structure 502 operates in a substantially similar manner as the support structure 402, but realizes different construction.

The support structure 502 can be formed of sufficiently rigid construction, for example, from steel, plastic, rubber or another sufficiently rigid material. The support structure 502 includes a series of openings 508 and a series of openings 509 through which the transfer belt 350 can be positioned to secure the support structure 502 against the torso of a person.

The support structure 502 includes a first and second pair of openings 510 spaced from one another through which one arm carrying member, such as arm carrying member 404 or another described above, can be attached. The support structure 502 also includes a first and second pair of openings 512 spaced from one another through which one arm carrying member, such as arm carrying member 406 or another described above, can be attached. The two pairs of openings 510 are spaced from the two pairs of openings 512 of the support structure 502.

One arm carrying member 404 can extend through the two pairs of openings 510 and the arm carrying member 406 can extend through the two pairs of openings 512 so that each arm carrying member 404, 406 is engaged with the support structure 502. For example, a first end 30 of the arm carrying member 404 could extend through a first opening 510 (uppermost opening, for example), over a portion of the person's arm, through a second opening 510 (second uppermost opening, for example), through a third opening 510 (third uppermost opening, for example), over a portion of the person's other arm, through a fourth opening 510 (lowermost opening, for example) before engaging a second end 32 of the arm carrying member 404 to support and secure both arms of the person against the support structure 502. A similar configuration can be used to position arm carrying member 406 through the openings 512, for example, but other configurations could be used as well.

The arm carrying members 404, 406 are removable from the support structure 502 for replacement, if necessary. Also, arm carrying members of different lengths can be substituted for one or both of the arm carrying members 404, 406 depending on the intended application.

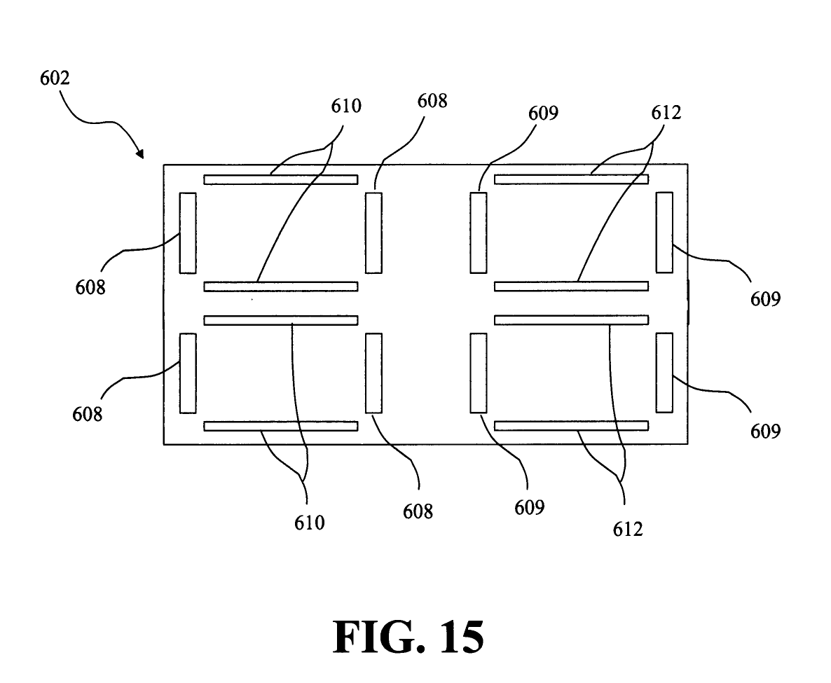

FIG. 15 shows another support structure 602, which is an alternative embodiment of the support structure 502 illustrated in FIG. 13. The support structure 602 operates in a substantially similar manner as the support structure 502, but realizes different construction. The support structure 602 is identical to the support structure 502, except that the support structure 602 provides an additional series of openings for receiving two transfer belts or axial straps to provide additional support to the support structure 602 and one or both arms of the person.

The support structure 602 includes a first series of openings 508 axially spaced from one another and a second series of openings 509 axially spaced from one another for receiving a first transfer belt or axial strap. Additionally, the support structure 602 includes another first series of openings 508 axially spaced from one another and another second series of openings 509 axially spaced from one another for receiving a second transfer belt or axial strap. The two series of openings 508 are spaced from one another and the two series of openings 509 are spaced from one another such that two transfer belts or axial straps can be used with each respective series of openings to provide additional support to the support structure 602 and one or both arms of the person.

The support structure 602 includes a first and second pair of openings 510 spaced from one another through which one arm carrying member, such as arm carrying member 404 or another described above, can be attached. The support structure 502 also includes a first and second pair of openings 512 spaced from one another through which one arm carrying member, such as arm carrying member 406 or another described above, can be attached. The two pairs of openings 510 are spaced from the two pairs of openings 512 of the support structure 502.

One arm carrying member 404 can extend through the two pairs of openings 510 and the arm carrying member 406 can extend through the two pairs of openings 512 so that each arm carrying member 404, 406 is engaged with the support structure 502. For example, a first end 30 of the arm carrying member 404 could extend through a first opening 510 (uppermost opening, for example), over a portion of the person's arm, through a second opening 510 (second uppermost opening, for example), through a third opening 510 (third uppermost opening, for example), over a portion of the person's other arm, through a fourth opening 510 (lowermost opening, for example) before engaging a second end 32 of the arm carrying member 404 to support and secure both arms of the person against the support structure 502. A similar configuration can be used to position arm carrying member 406 through the openings 512, for example, but other configurations could be used as well.

Support structure 502 or support structure 602 could be used in an immobilization device similar to device 400 described above. For example, either support structure 502 or support structure 602 could replace support structure 402 in device 400 although not explicitly shown.

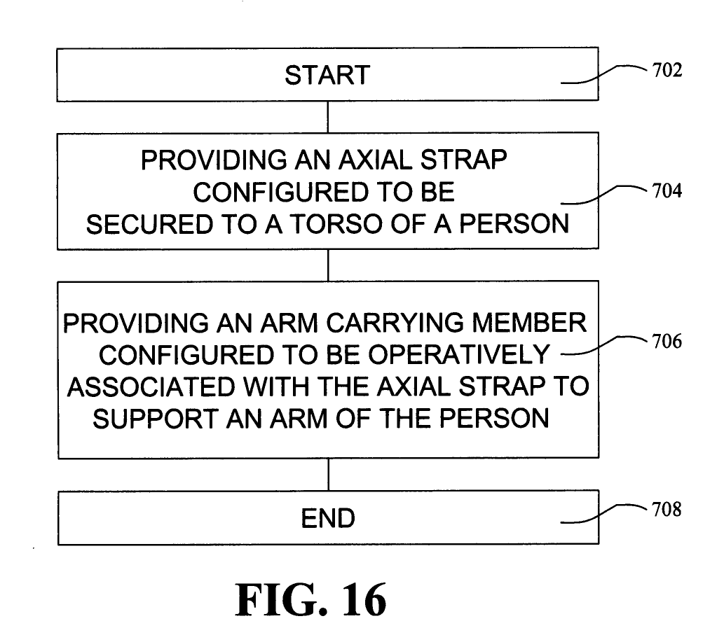

In each of the above-described embodiments of devices 10, 100, 200, 300 and 400 and support structures 402, 502 and 602, a person's arm is immobilized by positioning an axial strap 14 (or a transfer belt 350 in some embodiments) around a torso of the person 12. FIG. 16 shows a flow chart illustrating a method of immobilizing an arm of a person in accordance with the present invention. The method begins at 702. Upon positioning of the axial strap 14 or the transfer belt 350 at 704, a portion of the person's arm is supported with at least one arm carrying member 26, 28, 126, 128, 226, 404, 406 described above, which is operatively associated with the axial strap 14 or the transfer belt 350, at 704. The method ends at 706.

It will be appreciated that numerous modifications to and departures from the embodiments described above will occur to those having skill in the art. Thus, it is intended that the present invention covers the modifications and variations of the invention, provided they come within the scope of the appended claims and their equivalents.

1. A medical immobilizer device for a person, the medical immobilizer device comprising:
an axial strap having first and second ends configured to be releasably secured to one another around a torso of the person; and
an arm carrying member operatively associated with the axial strap and constructed and arranged to support a portion of at least one arm of the person when the axial strap is secured to the torso of the person.

2. A medical immobilizer device of claim 1, wherein the axial strap is a transfer belt.

3. A medical immobilizer device of claim 1, wherein the first and second ends include respective cooperative fastening structure configured to releasably secure the first and second ends to one another.

4. A medical immobilizer device of claim 3, wherein the cooperative fastening structure includes a loop fastener and a hook fastener configured to engage the loop fastener to releasably secure the first and second ends to one another.

5. A medical immobilizer device of claim 1, wherein the second end has cooperating fastening structure including a loop fastener thereon and the first end has cooperating fastening structure including a hook fastener thereon, the hook fastener being configured to engage the loop fastener to releasably secure the first and second ends to one another.

6. A medical immobilizer device of claim 1, wherein the arm carrying member has first and second end sections including respective cooperative fastening structure thereon configured to releasably secure the first and second end sections to one another.

7. A medical immobilizer device of claim 6, wherein the cooperative fastening structure includes a loop fastener and a hook fastener, the hook fastener being configured to engage the loop fastener to releasably secure the first and second end sections to one another.

8. A medical immobilizer device of claim 1, wherein the arm carrying member has a second end section with cooperating fastening structure including a loop fastener thereon and wherein the arm carrying member has a first end section with cooperating fastening structure including a hook fastener thereon, the hook fastener being configured to engage the loop fastener to releasably secure the first and second end sections to one another.

9. A medical immobilizer device of claim 1, wherein the arm carrying member is movably attached to the axial strap and constructed and arranged to be movable relative to the axial strap.

10. A medical immobilizer device of claim 1, wherein the arm carrying member is rotatably attached to the axial strap and constructed and arranged to be rotated relative to the axial strap to change the orientation of the arm carrying member relative to the axial strap.

11. A medical immobilizer device of claim 1, wherein the axial strap has a body portion between the first and second ends of the axial strap and wherein the body portion extends in an axial direction.

12. A medical immobilizer device of claim 12, wherein the arm carrying member is movably attached to the axial strap and constructed and arranged to be movable relative to the axial strap along the body portion thereof in the axial direction.

13. A medical immobilizer device of claim 1, further comprising an additional arm carrying member operatively associated with the axial strap and constructed and arranged to support a portion of at least one arm of the person when the axial strap is secured to the torso of the person.

14. A medical immobilizer device of claim 13, wherein at least one of the arm carrying member and the additional arm carrying member is movably attached to the axial strap and constructed and arranged to be movable relative to the axial strap.

15. A medical immobilizer device of claim 13, wherein at least one of the arm carrying member and the additional arm carrying member is rotatably attached to the axial strap and wherein at least one of the arm carrying member and the additional arm carrying member is constructed and arranged to be rotated relative to the axial strap to change the orientation thereof relative to the axial strap.

16. A medical immobilizer device of claim 1, further comprising a support structure operatively associated with the axial strap and the arm carrying member, the support structure being constructed and arranged to support a portion of at least one arm of the person in cooperation with the axial strap and the arm carrying member.

17. A medical immobilizer device of claim 16, wherein the support structure includes a first plurality of openings configured to receive the axial strap therethrough so that the support structure is movably attached to the axial strap.

18. A medical immobilizer device of claim 17, wherein the support structure includes a second plurality of openings configured to receive the arm carrying member therethrough so that the arm carrying member is movably attached to the support structure and to the axial strap.

19. A medical immobilizer device of claim 1, wherein the arm carrying member is constructed and arranged to support a portion of each arm of the person.

20. A medical immobilizer device for a person, the medical immobilizer device being configured for use with an adjustable belt having first and second ends, the medical immobilizer device comprising:
a support structure removably mounted to the adjustable belt; and
an arm carrying member removably mounted to the support structure and constructed and arranged to support a portion of at least one arm of the person.

21. A medical immobilizer device of claim 20, wherein the support structure includes a first plurality of openings configured to receive the adjustable belt therethrough so that the support structure is movably attached to the adjustable belt.

22. A medical immobilizer device of claim 21, wherein the support structure includes a second plurality of openings configured to receive the arm carrying member therethrough so that the arm carrying member is removably attached to the support structure and to the adjustable belt.

23. A medical immobilizer device of claim 20, further comprising an additional adjustable belt and an additional support structure removably mounted to the additional adjustable belt and configured to removably attach to the arm carrying member.

24. A method of immobilizing an arm of a person, the method comprising:
providing an axial strap configured to be secured only around a torso of the person; and
providing an arm carrying member operatively associated with the axial strap and constructed and arranged to support a portion of at least one arm of the person.
